(19) United States
(12) Patent Application Publication
(10) Pub. No.: US 2017/0360056 A1
(43) Pub. Date: Dec. 21, 2017

(54) FLAVOR MODULATION BY BIO-PROCESSING USING FLAVOR FORMING BACTERIA STRAINS

(71) Applicant: NESTEC S.A., Vevey (CH)

(72) Inventor: Marcel Braun, Konolfingen (CH)

(21) Appl. No.: 15/693,586

(22) Filed: Sep. 1, 2017

Related U.S. Application Data
(62) Division of application No. 13/995,432, filed on Jun. 18, 2013.

(30) Foreign Application Priority Data
Dec. 20, 2010 (EP) 10195845.2

(51) Int. Cl. A23C 9/127; A23C 9/13; A23C 9/123; C12N 1/20; C12P 7/24; C12P 7/04

(52) U.S. Cl. CPC A23C 9/1275; C12P 7/24; C12P 7/04; A23C 9/1236; C12N 1/20; A23C 9/1322; A23Y 2240/41

(57) ABSTRACT

A fermentation of a milk source with Lactococcus lactis subsp. lactis biovar diacetylactis (CNCM No. I-1962) to form a fermented milk product. The fermented milk product has a flavor and aroma. The fermented milk product can be in the form of a powder or a concentrate. The fermented milk product has applications in the food industry.

### PRIORITY CLAIM

The present application is a divisional application of U.S. patent application Ser. No. 13/995,432, filed on Jun. 1.8, 2013, which is a National Stage of International Application No. PCT/EP2011/073489, filed on Dec. 20, 2011, which claims priority to European Patent Application No. 10195845.2, filed Dec. 20, 2010, the entire contents of each of which are being incorporated herein by reference.

### FIELD OF INVENTION

The present invention relates to the generation of flavor and aroma in milk-based products. The generation of flavor and aroma in milk-based products is achieved using bacteria strains during fermentation of a milk source.

### BACKGROUND

Fermentation is a conversion of carbohydrates to organic acids or other compounds using bacteria strains.

Fermented milk products are major consumer products. Fermented milk products can be, for example, cheeses, buttermilks and yoghurts. Fermented milk products are manufactured by fermenting a milk source.

A milk source, for example milk, contains the carbohydrate lactose. During fermentation of the milk source the bacteria strains ferment the carbohydrate lactose to produce lactic acid. The production of lactic acid results in an acidification of the milk source during the manufacture of the fermented milk product. During fermentation of the milk source, other reactions may occur between other substances present in the milk source and the bacterial strains.

A fermentation of the milk source with bacteria strains is responsible for a generation of a flavor and aroma in the fermented milk products. Furthermore the fermentation of the milk source with the bacteria strains increases a shelf-life of the fermented milk products.

The bacteria strains used to ferment the milk source can be lactic acid bacteria strains. The lactic acid bacteria strains include Lactobacillus, Leuconostoc, Pediococcus, Lactococcus and Streptococcus; as well as the more peripheral Aerococcus, Carnobacterium, Enterococcus, Oenococcus, Sporolactobacillus, Teragenococcus, Vagococcus and Weiselia; these lactic acid bacteria strains belong to the order Lactobacillales.

An international patent application publication No. WO 2008/049581 by the Applicant Nestec SA is titled “Taste and flavour modulation by biotransformation in milk products”. The international patent application publication No. WO 2008/049581 discloses a method to promote a non-savory flavor in a food product.

An international patent application publication No. WO 02/085131 by the Applicant New Zealand Dairy Board is titled “Method of preparing savoury-flavoured products by fermentation of proteins”. The international patent application publication No. 02/085131 discloses a method for the manufacture of a savory flavored product from a source of protein using a combination of two distinct strains of bacteria. The source of protein may be a plant soy, wheat, rice, milk or whey. A first strain of bacteria is selected from the group Macrococcus, Micrococcus, Entercoccus, Staphylococcus, Brevibacterium, Anthrobacter and Corynebacterium, preferably Macrococcus caseolyticus. A second strain of bacteria is selected from the lactic acid bacteria-Lactococcus, Lactobacillus, Pediococcus or Leuconostoc. The savory flavored product may be combined with other ingredients to form products such as cheese, protein-water gels, yoghurts, creams, custards, sauces and confectionary products.

An international patent application publication No. WO 02/00845 by the Applicant Nizo Food Research is titled “Enhanced flavour production in or relating to food by cultivation of various food grade micro-organisms”. The international patent application publication No. WO 02/00845 discloses new mixed cultures of two or more micro-organism strains wherein at least one of the micro-organism strains which are comprised in the mixed culture is individually selected on the basis of its ability to perform part of an enzymatic pathway, and the two or more selected micro-organism strains together form a complete pathway towards a desired flavor component. The mixed culture is a culture for the production of a fermented product, such as yogurt or cheese or sausage. The two or more micro-organism strains are preferably co-cultivated. Particular and preferred embodiments are starter cultures for the manufacture of cheese. The mixed culture comprising a combination of various Lactoccocus strains and a combination of a Brevibacterium strain and a Staphylococcus strain, respectively.

The article “Characterisation of a malty-compound producing Lactococcus lactis subsp. lactis biovar. diacetylactis C1 strain isolated from naturally fermented milk” by Mutukumira et al. (2009) Milchwissenschaft 64(1) pp. 26-29, relates to a strain that produced acceptable fermented milk to a sensory panel despite the presence of a slight malty flavor.

The article “Production of fermented milk using a malty compound-producing strain of Lactococcus lactis subsp. lactis biovar. diacetylactis, isolated from Zimbabwean naturally fermented milk” by Narvhus et al. (1998) Internat. J. Food Microhiol. 41, pp. ‘73-80, relates to the preparation of fermented milk from several milks, using strain a strain of Lactococcus lactis subsp. lactis biovar. diacetylactis isolated from Zimbabwean naturally fermented milk.

However, due to a number of the lactic acid bacteria strains and their interactions with individuals, a selection of certain lactic acid bacteria strains to produce certain flavors and aromas in the fermented milk products is not predictable.

Fermented milk products have a wide variety of flavors and aromas depending upon the milk source and the lactic acid bacteria strains used to ferment the milk source.

There is a need to provide methods and lactic acid bacteria strains that are responsible for specific flavors and aromas in the fermented milk products.

Furthermore, artificial additives are negatively perceived by the consumer. There a need to provide flavor and aromas in a natural way that avoids artificial additives.

There is also a need to provide flavor and aromas which can be used in a wide range of foods.

There is thus a need to overcome the aforementioned problems in the art.

### SUMMARY

In an aspect the present invention relates to a method for the manufacture of a fermented milk product. The fermented milk product has at least a malty flavor and aroma. The method comprises providing a milk source, forming an amino acid supplemented milk source, to the amino acid supplemented milk source is added a Lactococcus lactis subsp. lactis biovar diacetylactis (CNCM No. I-1962) to form a mixture. The mixture is then fermented to manufacture the fermented milk product.

In a further aspect the present invention relates to a fermented milk product with at least a malty flavor and aroma obtained by the aforementioned method.

In a further aspect the present invention relates to a product for consumption by a mammal comprising the fermented milk product with at least a malty flavor and aroma.

In a further aspect the present invention relates to a food product comprising a lactic acid bacterium, Lactococcus lactis subsp. lactis biovar diacetylactis (CNCM No. I-1962) any one of 2-methylpropanal, 2/3-methylbutanal, phenylacetaldehyde and 2/3-methylbutanol.

In a further aspect the present invention relates to a use of a lactic acid bacterium, Lactococcus lactis subsp. lactis biovar diacetylactis (CNCM No. I-1962) to impart at least at least a malty flavor and aroma to a milk source.

The present inventors were surprised to find that a lactic acid bacterium, Lactococcus lactis subsp. lactis biovar diacetylactis (CNCM No. I-1962) imparts such flavor and aroma to the fermented milk products.

### DETAILED DESCRIPTION

For a complete understanding of the present invention and the advantages thereof, reference is made to the following detailed description of the invention.

It should be appreciated that various aspects of the present invention are merely illustrative of the specific ways to make and use the present invention.

The various aspects of the present invention can he combined with other aspects of the present invention and do not limit the scope of the invention when taken into consideration with the claims and the following detailed description.

The present invention concerns fermented milk products. The fermented milk products are manufactured by a fermentation of a milk source with a lactic acid bacterium to provide flavor and aroma to the fermented milk products.

The lactic acid bacterium is Lactococcus lactis subsp. lactis biovar diacetylactis. The lactic acid bacterium, Lactococcus lactis subsp. lactis biovar diacetylactis NCC 2415 was deposited in 1997 with the Institut Pasteur—Collection Nationale de Cultures de Mico-organisme (CNCM) with a CNCM No. I1-1962.

The milk source can be any type of milk, such as cow milk, sheep milk, goat milk and buffalo milk or any mixtures thereof. The milk source may be UHT-treated milk, pasteurized milk or non-pasteurized milk. The milk source may be full fat milk, a skimmed milk or semi-skimmed milk. Furthermore the milk source may be a fresh milk, recombined milk and milk containing vegetable fat and any mixtures thereof.

A conversion of amino acids into volatile flavor and aroma compounds plays an important role in food technology. The conversion of amino acids into volatile flavor and aroma compounds can be achieved by the lactic acid bacterium. Therefore there is a need to supplement the milk source with amino acids. That is to say that amino acids are supplemented to the milk source in addition to any amino acids that are naturally present in the milk source. In order to supplement the milk source at least one of an amino acid, a protease or a peptidase or any mixture thereof is added to the milk source. The amino acids are at least one of L-phenylalanine, L-leucine, L-isoleucine, L-valine. It is preferable that the peptidases is an exo-peptidases applied as enzyme preparation (for example produced by Aspergillus oryzae, Aspergillus sojae, Rhizophus oryzae, Bacillus subtilis, Ananas comosus) or as microbial strains with exoproteolytic activity (for example L. helveticus, S. thermophilus, L. plantarum, L. lactis subspecies). The peptidases or proteases release amino acid(s) such as L-phenylalanine, L-leucine, L-isoleucine, L-valine by an interaction with peptides and proteins naturally present in the milk source.

The amino acids are converted into volatile flavor and aroma compounds which are honey-like, malt-like or chocolate-like volatile flavor and aroma compounds.

The peptidases or proteases can be added without the amino acid(s) or with the amino acids. The at least one of the amino acid, the protease or the peptidase or any mixture thereof are added to the milk source in amount of 0.01 to 5 wt %, preferably 0.01-2 wt %, more preferably in an amount of 0.03-1.0 wt %, most preferably 0.05-0.3 wt %.

To the supplemented milk source is added the lactic acid bacterium, Laciococcus lactis subsp. lactis biovar diacetylactis (CNCM No. I-1962) to form a mixture.

Lipase can also he added to die milk source or the mixture to produce a desired flavor and aroma in the fermented milk products. Lipase hydrolyses fats in the milk source to form for example di-glycerides, monoglycerides and free fatty acids or any mixtures thereof. The di-glycerides, monoglycerides and free fatty acids impart a cream-like flavor to the fermented milk product. Lactase can also he added to the milk source or the mixture to produce desired flavor and aroma in the fermented milk products. Lactase hydrolyses the disaccharide lactose in the milk source into galactose and glucose. Glucose and galactose are used as flavor precursors for caramel-like and sweetened condensed milk-like flavor formation.

If the milk source is non-pasteurized, the milk source or the supplemented milk source may be pasteurized, subjected to ultra-high temperature treatment (UHT-milk) or sterilized under conditions known in the art. The pasteurization, ultra-high temperature treatment and sterilization is carried out in a temperature range of 70° C. to 150° C. for a time of between 2 s to 20 min. Alternatively, the milk source may be heat-treated prior to being the supplemented milk source.

The mixture of the supplemented milk source and the lactic acid bacterium (Lactococcus lactis subsp. lactis biovar diacetylactis) is then fermented to manufacture the fermented milk product with the malty flavor and aroma. The fermentation is allowed to take place for between 6 and :24 hours at a temperature of approximately 30° C.

Optionally, fermentation improving cofactors such as alpha-ketoglutarate, manganese or magnesium salts may also be added prior to the fermentation.

Depending upon the milk source it is to be appreciated that the fermented milk product with a malty flavor and aroma can be in the form of slurry (yogurt like) or a liquid. The fermented milk product can be further dried or concentrated.

The fermented milk product with the malty flavor and aroma can be dried, preferably by spray-drying and then converted into a powder.

Fermented milk product with the malty flavor and aroma. can have applications in food products and during a manufacture of food products. For example, the powder with the malty flavor and aroma can have applications in the beverage industry to impart the malty flavor and aroma to beverages. For example, the powder with the malty flavor and aroma can have applications in the food industry to impart the malty flavor and aroma to foodstuffs.

### EXAMPLES

The manufactured fermented milk products were analyzed by an electronic nose based on mass spectrometry and gas chromatography coupled to mass spectrometry (GC-MS).

Analysis with electronic nose based on mass spectrometry is a direct analysis method wherein the fermented milk product is placed directly into the ion source without the need for separation procedures and is therefore time-saving. A determination of volatiles from such a resultant mass spectra contains limited information for the identification of aroma components. Unequivocal identification of the single compounds present is not possible without prior separation and selective fragmentation i.e. GC-MS.

Gas chromatography coupled to mass spectrometry (GC-MS) provides the necessary separation and detection of volatiles. GC-MS is used for obtaining MS fragments belonging to a specific aroma component. The unambiguous identification of the molecules by GC-MS in combination with olfactometry is mandatory for analyzing volatiles with a specific odor.

Commonly used extraction methods for the isolation of volatiles from fermented milk products are vacuum distillation followed by solvent extraction, purge and trap (PT) and headspace techniques such as headspace solid-phase micro extraction (HS-SPME). The purge and trap (PT) and headspace techniques methods identify volatiles with different yield performances, but with comparable reproducibility. PT appeared to be a more sensitive whereas SPME is a more rapid and less expensive technique.

The reagents where used, were used as received without prior treatment unless otherwise stated.

### Example 1

### A-Reactivation of Lactic Acid Bacterium

The lactic acid bacterium, Lactococcus lactis subsp. lactis biovar diacetylactis (CNCM No. I-1962) in ampoules was reactivated with 1 ml reconstituted milk under sterile conditions, transferred in sterile glass tubes containing 9 ml reconstituted milk and incubated aerobically at 30° C. for 24 h in the dark.

The lactic acid bacterium, Lactococcus lactis subsp. lactis biovar diacetylactis (CNCM No. I-1962) were stored at 6° C. for two weeks and subsequently inoculated at 0.5% ((v/v) 0.05/10 ml medium) in a culture.

The culture was M17× (M17 Terzaghi Bouillon, Merck 1.15029 and 5 g/1 glucose (Merck 8342). After the growth phase (3 days) the flasks were stored at 6° C. to form a reactivated lactic acid bacterium.

Alternatively the culture can be skimmed milk.

### B Milk Source Supplementation with Amino Acids

An amino acid solution of 1.00 mM L-phenylalanine (Fluka, Buchs, Switzerland) (1.65 g/100 ml), L-leucine (Merck, Darmstadt, Germany) (1.31 g/100 ml), L-isoleucine (Merck, Darmstadt, Germany) (1.31 g/100 ml), L-valine (Merck, Darmstadt, Germany) (1.17 g/100ml) was dissolved in sterile water. The amino acid solution was filtrated through a pore size of 0.45 μm (Schleicher & Schuell, Whatmann, FP 30/0.45 μm, 7 bar max. CA-S). 500 μl of the amino acid solution was added to 4.5 ml UHT-milk (dilution 1:10) to obtain a supplemented milk source with a final concentration of 10mM in 5.0 ml.

### C-Fermentation

Fermentation in UHT-milk was performed by two approaches (I-II).

I: Index (Inside needle dynamic extraction; Hamilton) headspace sampling of volatile compound fragments in non-supplemented UHT-milk.

II. Tenax (accumulation absorbens, Marin-Epagnier, Switzerland) headspace sampling of volatile compounds in supplemented milk source UHT-milk (10 mM L, leucine, L-isoleucine, L-valine and L-phenylalanine).

An aliquot of 50 μl of the reactivated lactic acid bacterium was transferred in 5 ml supplemented milk source UHT milk (1% inoculation) under sterile conditions and incubated at 30° C. aerobically for 16-24 hours in the dark. An addition of 2.8 g NaC1 into the headspace vials helped to expel the volatiles from the fermented milk product into the headspace to get more intense release of the volatiles.

A electronic nose detected the volatile compound fragments at a range of m/z 40-100 for the experiment with non-supplemented UHT-milk (i.e. no amino acids) and at m/z 10-160 for the experiment with supplemented UHT-milk (i.e. with amino acids).

Principle component analysis (PCA) was calculated using the software program “The Unscrambler” (version 9.7). The results were calculated with logarithmised raw data and exclusion of the water and milk blanks. The calculations were done with all variables (MS fragments) included to group the strains in relation to similar MS-fragment patterns and abundance of compounds.

### D-Electronic Nose Measurements

Analysis of the ferments milk product by the electronic nose measurements in supplemented milk source UHT-milk was conducted. II: Tenax headspace measurement with 10 mM Leu, Lle, Val and Phe supplemented UHT-milk. GC-MS fragments [M]⁺were 27, 29, 43, 45, 60, 70, 86, 87, 88, 91, 103 and 120.

### E-pH and Redox Potential

A ph of the fermented milk product was determined to be 4.3 with a redox potential of 50 mV.

### F-Sensory Assessment of Fermented Milk Product

After fermentation the glass vials were kept close until sensory evaluation started. Seven persons attended the sensory assessment of the fermented milk product. The sensory assessment was based on the following attributes, scoring is noted with a X. A blank sample (incubated milk) was given as a reference. In order to test the influence of the amino acids samples were also prepared without the amino acids (addition of sterile water only) and presented to the panel. The results are shown below, wherein an X indicated a sensory perception of the fermented milk product.

| | |
|---|---|
| Buttery | — |
| Bitter/-almond | XX |
| Flower-like | X |
| Bread-like | XXXX |
| Creamy | X |
| Caramel | — |
| Strawberry | — |
| Fresh | — |
| Fruity | — |
| Yeast | X |
| Honey | X |
| Yoghurt | — |
| Cheesy | — |
| Milky | — |
| Malty | XXXX |
| Almond | XX |
| Nutty | X |
| Paper-like | — |
| Sweet | X |
| Acidic | X |
| Salty | — |
| Vanilla | — |

The results of the sensory assessment of fermented milk product demonstrate that the fermented milk product had a malty bread like flavor aroma. Other experiments have shown that by selection of the amino acid(s) different flavors and aromas can he generated.

### Example 2

### A-Reactivation of Lactic Acid Bacterium

A reactivation of lactic acid bacterium was carried out according to Example 1.

### B-Milk Source Supplementation with Amino Acids

An amino acid solution L-leucine (Merck, Darmstadt, Germany) (1.31 g/100 ml), L-isoleucine (Merck, Darmstadt, Germany) (1.31 g/100 ml), L-phenylalanine (Fluka, Buchs, Switzerland) (1.65 g/100 ml) was dissolved in sterile water. The amino acid solution was filtrated through a pore size of 0.45 μm (Schleicher & Schuell, Whatmann, FP 30/0.45 μm, 7 bar max. CA-S). 500 μl of the amino acid solution was added to 4.5 ml UHT-milk (dilution 1:10) to obtain a supplemented milk source with a final concentration of 10 mM in 5.0 ml.

### C-Fermentation

Fermentation in UHT-milk was performed by two approaches (I-II).

I: Index (Inside needle dynamic extraction; Hamilton) headspace sampling of volatile compound fragments in non-supplemented UHT-milk.

II: Tenax (accumulation adsorbens, Marin-Epagnier, Switzerland) headspace sampling of volatile compounds in supplemented UHT-milk (10 mM L-leucine, L-isoleucine, L, valine and L-phenylalanine).

An aliquot of 500 μl of the reactivated lactic acid bacterium was transferred in 5 ml supplemented milk source UHT milk (2% inoculation) under sterile conditions and incubated at 30° C. aerobically for 16 hours in the dark. An addition of 2.8 g NaCl into the headspace vials helped to expel the volatiles from the fermented milk product into the headspace to get more intense release of the volatiles.

The electronic nose detected the volatile compound fragments at a range of m/z 40-100 for the experiment with non-supplemented UHT-milk (i.e. no amino acids) and at m/z 10-160 for the experiment with supplemented UHT-milk (i.e. with amino acids).

Principle component analysis (PCA) was calculated using the software program “The Unscrambler” (version 9.7). The results were calculated with logarithmised raw data and exclusion of the water and milk blanks. The calculations were done with all variables (MS fragments) included to group the strains in relation to similar MS-fragment patterns and abundance of compounds.

### D-Electronic Nose Measurements

Analysis of the ferments milk product by the electronic nose measurements in supplemented UHT-milk was conducted. II: Tenax headspace measurement with 10 mM Leu, Ile, Val and Phe supplemented UHT-milk. GC-MS fragments [M]⁺were 27, 29, 43, 45, 60, 70, 86, 87, 88, 91, 103 and 120.

### E-pH and Redox Potential

A ph of the fermented milk product was determined to be 4.78.

### F-Sensory Assessment of Fermented Milk Product

After bacterial fermentation the glass vials were kept close until sensory evaluation started. Ten persons attended the sensory assessment of the fermented milk product. The sensory evaluation was a taste evaluation in order to gain information on the in mouth-effect and taste of the obtained fermented milk products.

In each case the samples were pasteurized (85° C. for 15 min in a water bath) and diluted to 1% in UHT milk (at a temperature 20-25° C.). The results shown below detail the inferences of the panelists.

| | Blank | |
|---|---|---|
| Panelist | Incubated UHT-milk | Fermented milk product |
| 1 | Slightly milky | Malty, Ovomaltine |
| 2 | Milky, UHT-milk | Strong malty, |
| | Slightly Sour | Ovomaltine-like, |
| | | sweetish, |
| | | slightly chocolate-like |
| 3 | Milky, Slight | Yeasty |
| | almond, | |
| | Bitter | |
| 4 | Milky | Malted |
| 5 | Milky, Cooked | Malted |
| 6 | Milk powder, Sweet | Malt with honey note |
| 7 | Milky, Fatty, | Malty cereals |
| | Buttery, Sweet | |
| 8 | — | Malty |
| 9 | — | Malty, Honey |
| 10 | — | Old, Stale. |

The results of the sensory assessment of fermented milk product demonstrate that the fermented milk product had a predominantly malty like flavor aroma with a twist of honey-chocolate like flavor aroma.

The malty note can be attributed to the presence of 2-methylpropanal, 2/3-methylbutanal, phenylacetaldehyde and 2/3-methylbutanol as determined by the electronic nose measurements.

Having thus described the present invention in detail, it is to be understood that the detailed description is not intended to limit the scope of the invention thereof.

What is desired to be protected by letters patent is set forth in the following claims.

1: A method for the manufacture of a fermented milk product with at least a malty flavor and aroma, the method comprising:
providing a milk source,
forming an amino acid supplemented milk source,
adding to the amino acid supplemented milk source a Lactococcus lactis subsp. lactis biovar diacetylactis (CNCM No. I-1962) to form a mixture; and
fermenting the mixture to produce the fermented milk product.

2: The method according to claim 1, wherein forming the amino acid supplemented milk source comprises the addition of at least one component selected from the group consisting of an amino acid, a protease and a peptidase to the milk source.

3: The method according to claim 1 comprising drying the fermented milk product and forming a powder.

4: The method according to claim 1 comprising concentrating the fermented milk product and forming a fermented milk product concentrate.

5: The method according to claim 1, wherein the milk source is selected from the group consisting of full fat milk, skimmed milk, semi-skimmed milk, fresh milk, recombined milk, cream, buttermilk, whey and milk containing vegetable fat.

6: The method according to claim 1, wherein the amino acid is selected from the group consisting of L-phenylalanine, L-leucine, L-isoleucine, and L-valine.

7: The method according to claim 1, wherein the peptidase is an exo-peptidase.

8: The method according to claim 1, wherein the protease is an exo-protease.

9: The method according to claim 1 comprising an addition of at least one of a lipase enzyme and a lactase enzyme to the milk source.

10: The method according to claim 1 comprising adding a fermentation co-factor to the milk source.

11: A method for imparting at east a malty flavor and aroma to a milk source, the method comprising:
adding a lactic acid bacteria Lactococcus lactis subsp. lactis biovar diacetylactis (CNCM No. I-1962) to the milk source.
